(19) United States
(12) Patent Application Publication
(10) Pub. No.: US 2022/0298571 A1
(43) Pub. Date: Sep. 22, 2022

(54) SUPER HLA TYPING METHOD AND KIT THEREOF

(71) Applicant: Malali GOWDA, Yelahanka, Bengaluru (IN)

(72) Inventor: Malali GOWDA, Yelahanka, Bengaluru (IN)

(73) Assignee: Malali GOWDA, Yelahanka, Bengaluru (IN)

(21) Appl. No.: 17/250,113

(22) PCT Filed: May 28, 2019
(86) PCT No.: PCT/IB2019/054409

§ 371 Date: Nov. 30, 2020

(30) Foreign Application Priority Data
May 29, 2018 (IN) 201741042776

(51) Int. Cl. C12Q 1/6881; C12Q 1/686

(52) U.S. Cl. CPC C12Q 1/6881; C12Q 1/686

(57) ABSTRACT

The present invention method for super Human Leukocyte Antigen (HLA) Typing, gene sequencing and analysis comprising: (a) designing and synthesizing the set of forward and reverse primers for full length amplification of HLA gene class I (HLA-A, B, C) and class II (HLA-DQBI, DRBI, DPBI)selected from group consisting of SEQ ID 1 to 24; (b) amplification of the test sample HLA gene by using the set of primer synthesized at step (a) to obtain PCR amplicon; (c) separation of the PCR amplicon obtained at step (b); (d) sequencing of the separated amplicon of step (c); and (e) the sequences thus generated analyzed by matching with HLA gene from 5′ UTR to 3′ UTR.

### RELATED APPLICATIONS

This application is a national phase application of and claims priority under 35 U.S.C. § 371 of PCT IB Patent Application Serial No. PCT/162019/054409 filed on May 28, 2019 and titled SUPER HLA TYPING METHOD AND KIT THEREOF. The content of this application is incorporated herein by reference.

### FIELD OF THE INVENTION

The present invention relates to sequencing of full-length Human Leukocyte Antigen (HLA) typing for class I and class II genes. It is an object of the present invention to provide accurate full length sequence of HLA genes, class I (HLA-A, HLA-B, HLA-C) and class II (HLA-DRBI, HLA-DQBI and HLA-DPBI) genes using next generation sequencing technologies. It is an object of the present invention to provide HLA kit to detect allele sequences of class I and class II genes. The invention also relates to a method and kit for full length HLA gene sequences.

### BACKGROUND OF THE INVENTION

The major histocompatibility complex (MHC) consists of immunologically important genes that encode for HLA proteins, which help the immune system to recognize foreign antigens (pathogens and allergens). HLA genes are present on short arm of chromosome 6, which have been extensively studied in human population, HLA genes being the most polymorphic loci in the human genome.

The HLA class I and class II genes are a component of the human MHC. The class I genes consist of the three classical genes encoding the major transplantation antigens HLA-A, HLA-B and HLA-C. The class II genes consist of three classical genes encoding the major transplantation antigens, HLA-DR, HLA-DQ and HLA-DP.

HLA system plays an important role in organ transplantation, infectious diseases and genetic disorders. HLA genes encode cell membrane proteins, which recognize the self or non-self of cell types.

HLA genes have been associated with cell type identification in organ transplantation, many autoimmune diseases, cancer, drug hypersensitivity and allergy responses, infectious diseases (viral and bacterial), anthropological studies (population genetics), vaccines and fingerprinting (paternity and forensic study) etc. In addition, numerous HLA alleles have been identified as signatures of risk indices of susceptibility to diseases, biomarkers of drug hypersensitivities, predictors of graft outcome post transplantation and tags of ethnic diversity.

There are reports in the patent and non-patent literature which reported for the sequencing of HLA genes, the important ones include US 2016/0208326 which discloses a method and kit for. This method for the DNA typing of HLA, which is characterized by comprising: (1) a step of preparing a set of primers which can respectively anneal specifically to an upstream region and a downstream region of each of HLA-A, HLA-B, HLA-C, HLA-DQA1 HLA-DQB1 HLA-DPAI and HLA-DPBI gene in the nucleotide sequence for the human genome, and a set of primers which can respectively anneal specifically to exon-2 and a 3′-side non-translated region in HLA-DRB I; (2) a step of carrying out the PCR amplification of a sample to be tested (DNA) using the sets of primers; (3) a step of determining the nucleotide sequence for a PCR—amplified product: and (4) an optional step of carrying out the homology search in a database. This invention may not provide complete gene sequence of class II HLA gene, DRB1. This invention uses short read method of NGS using Roche 454 sequencing technology. Short reads stitched together by bioinformatics tools, which may create inaccurate alleles during allele phasing. Our invention provides complete gene sequence for class I (HLA-A, HLA-B, HLA-C} and class II (HLA-DRBI, HLA-DQBI and HLA-DPBI).

US 2017/0029885 disclose a method and kit for the DNA profiling of HLA genes using a high-throughput massively parallel sequencer. A method for the DNA profiling of HLA genes, said method being characterized by including: (1) a step for preparing a primer set that anneals specifically to exon 4 and intron 1 and includes exon 2 and exon 3 of at least one target gene selected from the group consisting of HLA-DRBI, HLA-DRB3, HLA-DRB4, HLA-DRBS, HLA-DQBI and HLA-DPBI in the base sequence of the human genome: (2) a step for amplifying a sample (DNA) by PCR using the primer set; (3) a step for determining the base sequence of the amplified PCR product: and (4) a step for carrying out a homology search against a database. This invention may not provide complete gene sequence of class I and class II HLA genes. This claim used short read next generation sequencing method, which fragment sequences and fuse reads of sequences to generate phasing of alleles. This may create inaccurate phased HLA alleles. Our invention provides complete gene sequence for class I (HLA-A, HLA-B, HLA-C) and class II (HLA-DRBI, HLA-DQBI and HLA-DPBI).

CN101962676A invention discloses a human leukocyte antigen HLA-A and HLA-B gene full-length sequencing method and an HLA gene sequencing and typing method. The HLA-A and HLA-B gene full-length sequencing method comprises the following steps of: a, performing PCR amplification on about 4 kb full-length sequences of HLA-A and HLA-B genes by using a pair of primers respectively; and b, cloning the amplification products to a pGEM-T easy vector, sequencing the full-length sequences by using ten walking sequencing primers in positive and negative directions respectively, and totally obtaining 38 allele 4.3 Kb full-length sequences of the HLA-A and 30 allele 3.7 Kb full- length sequences of the HLA-B. The HLA-A and HLA-B sequencing and typing method comprises the following steps of: performing PCR amplification on typing target areas of the HLA-A and HLA-B by using two pairs of primers respectively; and performing two directional sequencing on products by using fourteen sequencing primers respectively, wherein the HLA-DRBI and HLA-DQBI sequencing and typing method comprises the following steps of: amplifying sequences of second and third exons of DRB1 and DQB1 by adopting group specificity primers respectively; performing two directional sequencing on the second and third exons of the DRB1 by adopting eight group specificity primers and three sequencing primers; and performing two directional sequencing on the second and third exons of the DQBI by adopting four sequencing primers, respectively. This invention seems to be cumbersome because they adopted cloning strategy and Sanger sequencing by primer walking. The invention is only restricted to class I genes, HLA-A, B, and only second and third exons of HLA-DRBI and DQBI genes.

Danzer et. al. 2013 [Martin Danzer, Norbert Niklas, Stephanie Stabentheiner, Katja Hofer, Johannes Prem, Christina Sttickler, Rapid, Edeltraud Raml, Helene Polin and Christian Gabriel, BMC Genomics 2013, 14:221] discloses scalable and highly automated HLA genotyping using next-generation sequencing: a transition from research to diagnostics. This invention involves selective amplification of exonic and intronic regions of class I (HLA-A, B, C) and class II (HLA-A DRBI, DQBI, DPBI) genes.

Clark et. al. 2016 [Peter M. Clark, Jamie L. Duke, Deborah Ferriola, Valia Bravo- Egana, Tunde Yago, Aniqa Hassan, Anna Papazoglou, and Dimitri Monos, Clinical Chemistry 62:12, 1630-1638 (2016)] discloses generation of full-length class I human leukocyte antigen gene consensus sequences for novel allele characterization. This invention provides full length amplification of only class I (HLA-A, HLA-B, HLA-C) genes. Wittig et. al., 2015 [Michael Wittig, Jarl A. Anmarkrud, Jan C. K'assens, Simon Koch, Michael Forster,Eva Ellinghaus, Johannes R. Hov, Sascha Sauer, Manfred Schimmler, Malte Ziemann, Siegfried G·· org, Frank Jacob, Tom H. Karlsenand Andre Franke, Nucleic Acids Research, 2015, Vol. 43, No. 11 e70] “Development of a high-resolution NGS-based HLA-typing and analysis pipeline”. This invention uses target enrichment approach for high resolution HLA sequencing which may not provide complete gene sequence of class I HLA and class II HLA genes.

Yin et al., 2016 [Yuxin Yin, James H. Lan, David Nguyen, Nicole Valenzuela, Ping Takemura, Yung-Tsi Bolon, Brianna Springer, Katsuyuki Saito, Ying Zheng, Tim Hague, Agnes Pasztor, Gyorgy Horvath, Krisztina Rigo, Elaine F. Reed, Qiuheng Zhang, PLOS ONE I DOI:10.1371/journal.pone.0165810 Oct. 31, 2016] which discloses application of high-throughput next-generation sequencing for HLA typing on buccal extracted DNA: results from over 10,000 donor recruitment samples. This invention may not provide complete gene sequence of class I HLA and class II HLA genes.

Manish J et al. 2017 [Manish J. Gandhi, M D; Deborah Ferriola, B S; Yanping Huang, M D, PhD; Jamie L. Duke, PhD; Dimitri Monos, PhD, Arch Pathol Lab Med-Vol 141, June 2017] which discloses targeted next-generation sequencing for human leukocyte antigen typing in a clinical laboratory metrics of relevance and considerations for its successful implementation. This invention may not provide complete gene sequence of class I HLA and class II HLA genes.

Hosomichi et al., 2015 [Kazuyoshi Hosomichi, Takashi Shiina, Atsushi Tajima and Ituro Inoue, Journal of Human Genetics (2015) 60, 665-673] which discloses the impact of next-generation sequencing technologies on HLA research. Our invention provides complete gene sequence for class I (HLA-A, HLA-B, HLA-C) and class II (HLA-DRBI, HLA-DQBI and HLA-DPBI).

Larjo et. al., 2017 [Antti Larjo, Robert Eveleigh, Elina Kilpelainen, Tony Kwan, Tomi Pastinen, Satu Koskela and Jukka Partanen, Frontiers in Immunology frontiersin.nrg. December 2017 | Volume 8 | Article 1815] which discloses accuracy of programs for the determination of human leukocyte antigen alleles from next-generation sequencing data. This invention utilizes low and medium resolution techniques for HLA typing and analysis, which may not provide complete gene sequence of class I and class II HLA genes.

Conventionally, serological, sequence-specific oligonucleotide (SSO) and sequence-specific primer (SSP) and Sanger sequencing methods have been used to analyzing HLA loci for organ transplantation. These methods are based on low-resolution of HLA typing, which give ambiguity for transplantation donor finding.

Though above reports do HLA typing by sequencing either by Sanger or short read technologies. There are limitations or drawbacks in HLA typing and sequencing of full length HLA I and II and data analysis to find out haplotype of HLA alleles. The recent next generation sequencing (NGS) technologies have decreased the cost per base sequencing. However, the limitation of above inventions and publications produced data from short reads from NGS methods. This may not provide complete phasing and haplotype information of parental HLA alleles, thus creating confusion among clinicians and doctors for accurate HLA alleles matching between organ donor and recipient for transplantation. NGS technologies have potential advantages over the Sanger method by increasing HLA typing sequencing depth, speed, multiplexing, resolution and low cost per sample (Lind C, Ferriola D, Mackiewicz K, Heron S, Rogers M, et al. (2010). Next generation sequencing: the solution for high-resolution, unambiguous human leukocyte antigen typing. Hum Immunol 71: 1033-1042. Wittig M, Anmarkrud J A, Kassens J C, Koch S, Forster M, et al. (2015). Development of a high-resolution NGS-based HLA- typing and analysis pipeline. Nucleic Acids Res 43: e70). NGS allows clonal amplification for the region sequenced and can also get a high-resolution. NGS technologies are more sensitive to detect rare alleles as compared to Sanger sequencing.(Cost-efficient high-throughput HLA typing by MiSeq amplicon sequencing: Vinzenz Lange, Irina Bohme, Jan Hofmann, Kathrin Lang, Jtirgen Sauter, Bianca Schone, Patrick Paul, Viviane Albrecht, Johanna M Andreas, Daniel M Baier, Jochen Nething, Ulf Ehninger, Carmen Schwarzelt, Julia Pingel, Gerhard Ehninger, and Alexander H Schmidt, BMC Genomics. 2014; 15: 63).

The major problem in short read NGS technologies is that these may not resolve phasing of parental alleles. To overcome above limitations, there exists a need for development of a method and kit for full length DNA sequencing for class I and class II HLA genes and accurate allele segregation during analysis. The present invention inventor has developed an efficient novel method and a kit for Super HLA Typing, sequencing by long read third generation sequencing (Oxford Nanopore) technology. This method can sequence complete HLA class I and II genes including introns, exons and part of UTR regions of HLA genes. Our approach provides one-time solution with reduced cost per sample and subsequently assists in building the HLA database for population.

### SUMMARY OF THE INVENTION

The primary object of the present invention is the development of Super HLA Typing method for class I and class II genes by providing accurate full length sequence of HLA genes and alleles.

The other object of the present invention is the development of method for Super HLA Typing for class I and class II genes by providing accurate full length sequence of HLA class I (HLA-A, HLA-B, HLA-C) and HLA class II (HLA-DRBI, HLA-DOBI and HLA-DPBI) genes.

Another object of the present invention to develop the Super HLA Typing kit to detect allele sequences of HLA class I and class II genes.

The other object of the present invention is the development of method and kit for Super HLA Typing for class I and class II genes by providing accurate full length sequence of HLA genes by using long read sequencing technology, which is cost-effective method.

By the other object of the present invention is the development of method and kit for Super HLA Typing for class I and class II genes providing accurate full length sequence of HLA genes, which are easy to use with little technical expertise.

### BRIEF DESCRIPTION OF THE DRAWINGS

FIG. 1 is a diagrammatic representation of HLA-A primer location.

FIG. 2 is a diagrammatic representation of HLA-B primer location.

FIG. 3 is a diagrammatic representation of HLA-C primer location.

FIG. 4 is a gel image of PCR amplified products of HLA-A, B, C

FIG. 5 is a diagrammatic representation of HLA-DQBI primer location

FIG. 6 is a gel image of PCR amplified products of HLA-DQBI

FIG. 7 is a diagrammatic representation of HLA-DRBI primer location

FIG. 8 is a gelimage of PCR amplified products of HLA-DRBI

FIG. 9 is a diagrammatic representation of HLA-DPBI primer location

FIG. 10 is a gel image of PCR amplified products of HLA-DPBI.

FIG. 11 is a diagrammatic representation of method to capture and sequence full length HLAgenes

### STATEMENT OF THE INVENTION

An embodiment of the invention is directed to a method for Super HLA Typing, gene sequencing and analysis comprising:
(a) designing and synthesizing the set of forward and reverse primers for full length amplification of HLA genes class I (HLA-A, B, C) and class II (HLA-DQBI, DRBI, DPBI) selected from group consisting of SEQ ID 1 to 24;
(b) amplification of the test sample HLA gene by using the set of primer synthesized at step
   (a) to obtain PCR amplicon;
(c) separation of the PCR amplicon obtained at step (b);
(d) sequencing of the separated amplicon of step (c); and
(e) the sequences thus generated analyzed by matching with HLA gene from 5′ UTR (UnTranslated Region) to 3′ UTR (UnTranslated Region).

The primers in Super HLA Typing for HLA genes have been designed by using one of the longest sequences as “reference sequence”, other HLA gene sequences from IMGT were aligned to reference, and then most conserved region was selected for designing primer sequence. The amplification is carried out with long range Taq polymerase enzyme.

The set of primers for amplification of HLA-A gene selected are SEQ ID No. 1 and SEQ ID No 2.

The set of primers for amplification of HLA-B gene selected are SEQ ID No. 3 and SEQ ID No 4.

Method as claimed in claim 1 wherein the set of primers for amplification of HLA-C gene selected are SEQ ID No. 5 and SEQ ID No 6.

The set of primers for amplification of HLA-DRBI gene selected are SEQ ID No. 7 to SEQ ID No 12.

The set of primers for amplification of HLA-DQBI gene selected are SEQ ID No. SEQ ID No. 13 to SEQ ID No 18.

The set of primers for amplification of HLA-DPBI gene selected are SEQ ID No. SEQ ID No. 19 to SEQ ID No 24.

The obtained sequences in the method optionally were subjected for homology search in the database and BLAST (www.ncbi.nlm.nih.gov/BLAST) against NCBI database to find the best match.

Primers for HLA gene amplification, sequencing and analysis comprising the selected from group consisting of SEQ ID No. Ito 24.

Super HLA Typing kit for HLA gene sequencing and analysis comprising the set of primers for amplification of HLA genes selected from group consisting of SEQ ID No. I to 24 and integrated bioinformatics tools along with the allele calling pipeline.

The sequence listing is incorporated by reference:

### Name: MG_HLA_Primers_ST25

### Date of Creation: Jun. 22, 2021

Size: 5,648 bytes

### DETAILED DESCRIPTION OF THE INVENTION

The present disclosure relates to a method and the kit for Super HLA Typing for class I and class II genes.

Those skilled in the art will be aware that the present disclosure is subject to variations and modifications other than those specifically described. It is to be understood that the present disclosure includes all such variations and modifications. The below illustrations are for the understanding of the invention and should in no way be constructed to be limiting.

This invention used blood, buccal swab and saliva for HLA genes amplification using long range PCR.

Buccal sample was collected using ORACollect DNA swab kit (Ref. No OCR-I 00) from DNA Genotek Ontario, Canada. The DNA isolation was performed using the standardized ORACollect DNA buccal swab prepIT-L2P DNA extraction protocol (Cat No:Q-33130) as per the manufacturer's protocol. The extracted DNA was checked for quality using 0.8% agarose gel and quantified using Nanodrop and Qubit.

Also I tested saliva collection and DNA isolation from human samples using BGC (Bengaluru Genomics Center) DNA isolation kit (Cat. No: B10241). In this method, the intake of any food or drink was restricted for two hours for sample collection. Before collection of samples, subjects were asked to rinse mouth twice with 50 mL distilled water and asked them to drink 50 mL of water to reduce the microbial DNA. About 1 to 0.5 mL of saliva was collected by direct spitting of saliva in 15 mL tube that containing 1 mL of storage buffer. Saliva sample was mixed properly with storage buffer by inversion of tubes several times. DNA was isolated according to manufacturer's protocol and the extracted DNA was checked for the quality using 0.8% agarose gel and quantified using Nanodrop and Qubit.

Genomic DNA was extracted form 500 μL of blood using QIAamp DNA Blood Mini Kit (Cat No./ID: 51104) according to the manufacturer's protocol. The extracted DNA was checked for quality using 0.8% agarose gel and quantified using Nanodrop and Qubit.

PCR amplification of class I (HLA-A, B, C) and class II (HLA-DRBI, DQ131, DPBI) genes was performed using Roche's Kapa HiF Hotstart Readymix PCR Kit (Code: KK2601 07958927001). The primers used for amplification are as described in Table:1. PCR was performed in 10 μL reaction volume containing 60-IO0 ng of genomic DNA, 5 μL Kapa HiFi Hotstart Readymix, 0.5 μL of Forward and Reverse Primers (IOpm/μL). The cycling parameters for the PCR are as follows: 95° C. for 3 min (first denaturing step), 35 cycles of 98° C. for 20 sec, 65° C. for 15 sec, 72° C. for 7 min and 72° C. for 10 min (last extension step). The primers were designed in such a way that all the PCR primer pairs for class I and class II have common annealing temperature. The PCR reaction was carried out in Applied Biosystem's Veriti 96-well Thermal Cycler (Cat No: 4375786).

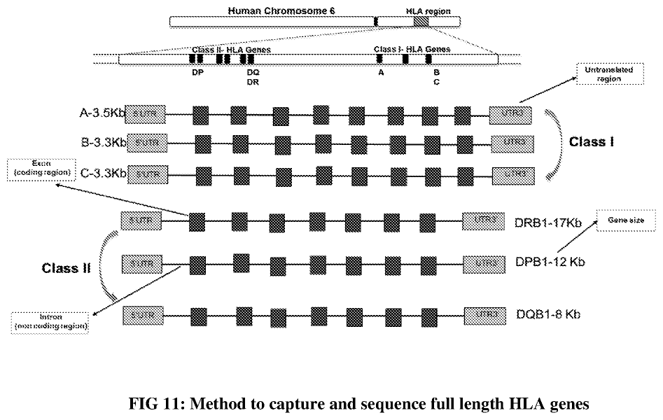

Details of gene structure of class I (HLA-A, B, C) and class II (HLA-DRBI, DQBI, DPB1) is depicted in FIG. 11.

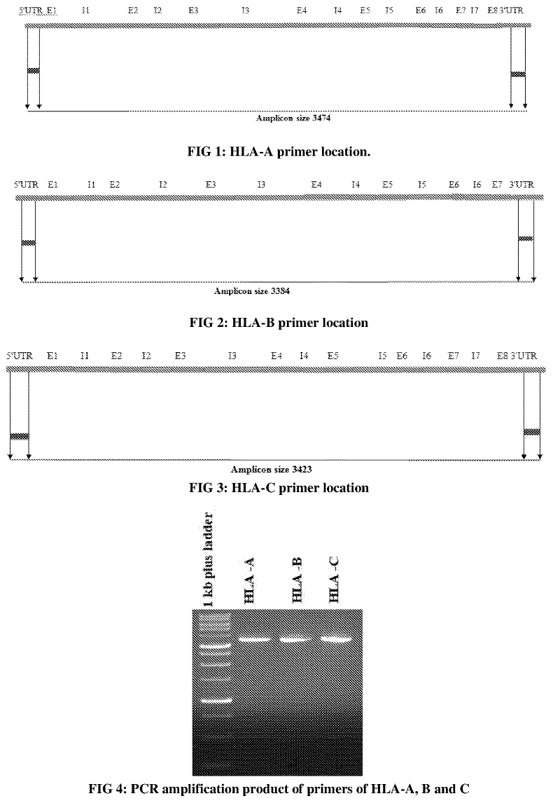

In accordance with the embodiments of the present invention, HLA-A gene was studied by designing specific primer sequences. The primer sequence location is depicted in FIG. 1. The amplified product of primer sequence is shown in FIG. 4. Then the forward primers to the 5′ UTRregion (12-30 bp) were designed, and reverse primers were designed from 3486 to 3507 bp. The said primer was synthesized using oligo synthesizer. These oligo bases were used to amplify HLA-A from the human blood and saliva samples. I obtained 3474-3488 bp of HLA-A amplicon as depicted in FIG. 4. This band was purified from 1% agarose gel and sequenced using third generation sequencing (Oxford Nanopore) method. Then the high quality sequences were BLAST (www.ncbi.nlm.nih.gov/BLAST) against NCBI and IMGT databases to find the best match. The PCR amplicons generated thus were matched to HLA-A gene from 5′ UTR to 3′ UTR (12-3507 bp) as expected.

Similarly, HLA-B gene was studied using specific primers (FIG. 2), PCR amplification (FIG. 4) and analysis. All the full length HLA-B alleles were (HLA-B gene IDs as shown in Table 2) downloaded from NCBI, then aligned all these sequences to reference sequence (NCBI ID: KX774745) to locate SNPs across the HLA-B gene length. Then designed the forward primers to the 5′ UTR region (20-41 bp), and reverse primers designed from 3369-3389 bp. Then primers were synthesized using oligo synthesizer. These oligo bases are used to amplify HLA-B from the human blood and saliva samples. I obtained 3366-3384 bp of HLA-B amplicon. This band was purified from 1% agarose gel and sequenced using third generation sequencing (Oxford Nanopore) method. Then high quality sequences were BLAST (www.ncbi.nlm.nih.gov/BLAST) against NCBI database to find the best match. The PCR amplicons thus generated matched HLA gene from 5′ UTR to 3′ UTR (20-3389) as expected.

HLA-C gene was studied using specific primers (FIG. 3), PCR amplification (FIG. 4), sequencing and analysis. All the full length HLA-C alleles were (HLA-C NCBI IDs as shown in Table 3) downloaded from NCBI database, then aligned all these sequences to reference sequence (NCBI ID: KX649940) to locate SNPs across the HLA-C gene length. Then designed the forward primers to the 5′ UTR region (19-42 bp), and reverse primers designed from 3415-3435 bp. Then primers synthesized using oligo synthesizer. These oligo bases are used to amplify HLA-C from the human blood and saliva samples. I obtained 3415-3423 bp of HLA-C amplicon. This band was purified from 1% agarose gel and sequenced using third generation sequencing (Oxford Nanopore) method. Then high quality sequences were BLAST (www.ncbi.nlm.nih.gov/BLAST) against NCBI sequence database to find the best match. The PCR amplicons thus generated matched to HLA gene from 5′ UTR to 3′ UTR(I9-3435).

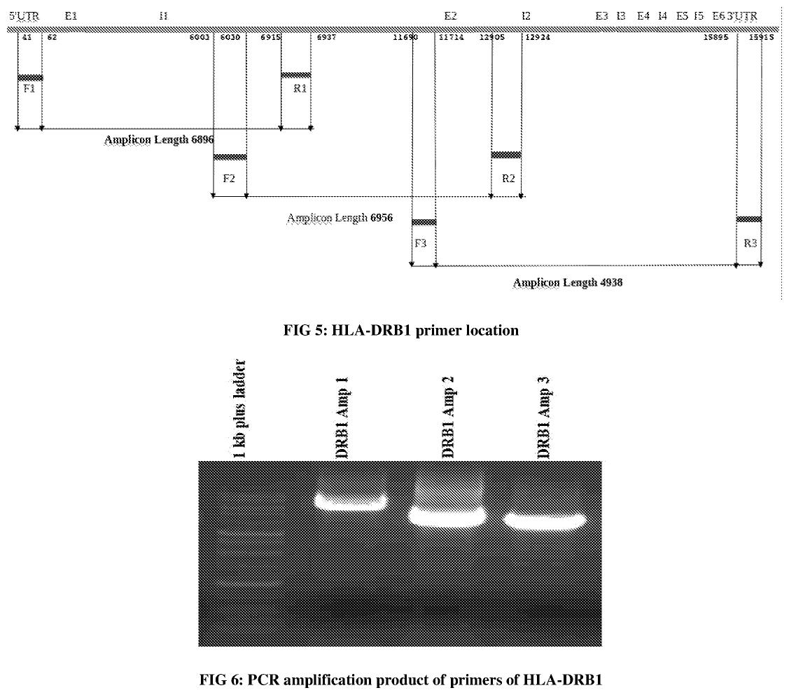

In accordance with the embodiments of the present invention, HLA-DQBI gene was studied by designing three specific primer sequences because of the large gene sequence (7230 bp). The primer sequences location is depicted in FIG. 5. Three sets of primers were designed in-order to cover the full length. Three amplicon sizes were 2936 (Forward: 56-84, Reverse: 2968-2991), 2638 (Forward: 2822-2845, Reverse: 5436-5459) and 1971 (Forward: 5141-5164, Reverse: 7090-7111) respectively. The said primers were synthesized using oligo synthesizer. These oligo bases were used to amplify HLA-DQB 1 from the human blood and saliva samples. I obtained 3474-3488 bp of HLA-DQBI amplicon as depicted in FIG. 4. This band was purified from 1% agarose gel and sequenced using third generation sequencing (Oxford Nanopore) method. Then the high quality sequences were BLAST (www.ncbi.nlm.nih.gov/BLAST) against NCBI and IMGT databases to find the best match. The PCR amplicons generated thus were matched to HLA-DQBI gene from 5′ UTR to 3′ UTR as expected.

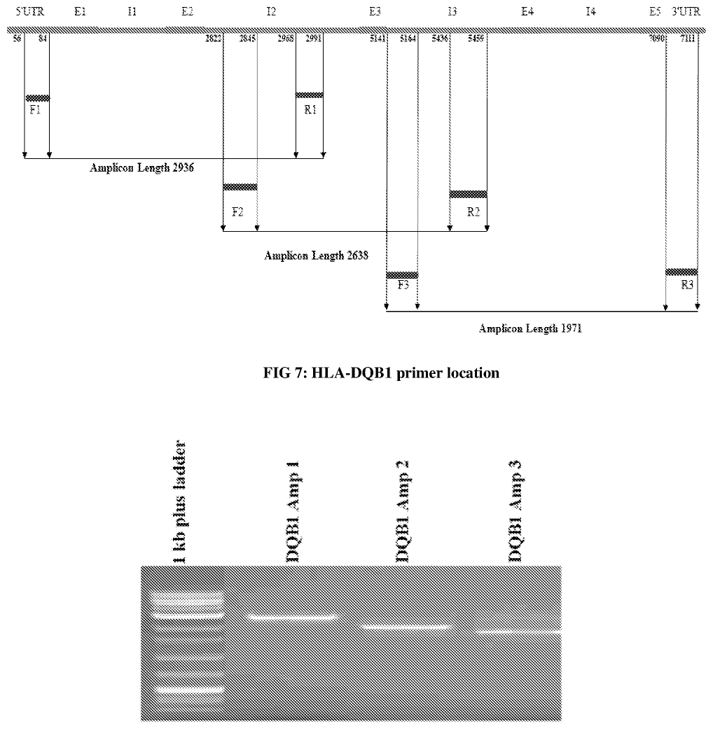

HLA-DRB 1 gene was studied using specific primers (FIG. 7), PCR amplification (FIG. 8), sequencing and analysis. All the full length HLA-DRBI alleles were downloaded from IMGT database, then aligned all these sequences to reference sequence (IMGT ID: HLA: HLA03486) to locate SNPs across the HLA-DRBI gene length. The total gene length is 16120 bp. Three sets of primers were designed in-order to cover the full length. The three amplicon sizes were 6896 (Forward: 41-62, Reverse: 6915-6937), 6956 (Forward: 6003-6030, Reverse: 12936-12959) and 4938 (Forward: 10977-10997, Reverse: 15895-15915) respectively. Then primers synthesized using oligo synthesizer. These oligo bases are used to amplify HLA-DRBI from the human blood and saliva samples. This band was purified from 1% agarose gel and sequenced using third generation sequencing (Oxford Nanopore) method. Then high quality sequences were BLAST (www.ncbi.nlm.nih.gov/BLAST) against NCBI and IMGT databases to find the best match. Our results showed that PCR amplicons matched to HLA gene from 5′ UTR to 3′ UTR (41-15915).

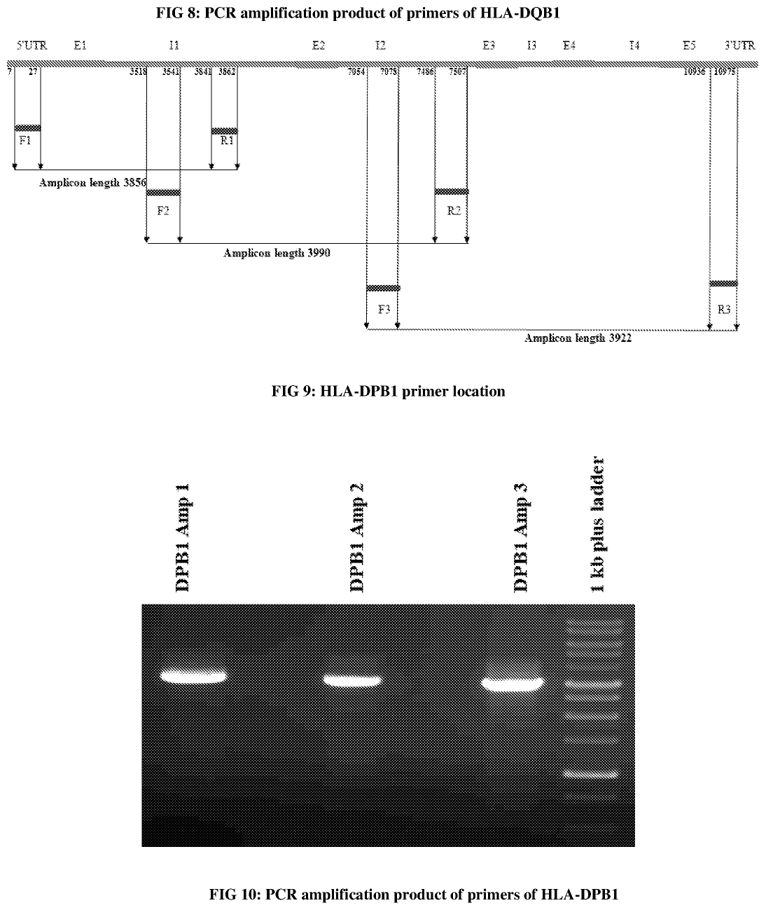

HLA-DPB I gene was studied using specific primers (FIG. 9), PCR amplification (FIG. 10), sequencing and analysis. All the full length HLA-DPBI alleles were (IMGT HLA-DPBI gene IDs is shown in Table 6) downloaded from IMGT database, then aligned all these sequences to reference sequence (IMGT ID: HLA: HLA00517) to locate SNPs across the HLA-DPBI gene length. The total gene length is 11532 bp. Three sets of primers were designed in-order to cover the full length. The three amplicon sizes were 3856 (Forward: 7-27, Reverse: 3841-3862), 3990 (Forward: 3518-3541, Reverse: 7486-7507) and 3922 (Forward: 7054-7078, Reverse: 10936-10975) respectively. Then primers synthesized using oligo synthesizer. These oligo bases are used to amplify HLA-DPBI from the human blood and saliva samples. This band was purified from 1% agarose gel and sequenced using third generation sequencing (Oxford Nanopore) method. Then high quality sequences were BLAST (www.ncbi.nlm.nih.gov/BLAST) against NCBI and IMGT databases to find the best match. Our results showed that PCR amplicons matched to HLA gene from 5′ UTR to 3′ UTR (7-10975).

### Long Range PCR Amplification

The amplification of full length HLA gene of class I and II was standardized using long range Taq DNA polymerase (Kapa Kit, Code: KK2601 07958927001). Since the amplicons size ranges from 2 kb to 7 kb, long range PCR amplification kit was used for amplification. The amplified PCR product of class I and class II were purified using gel based purification method (QIAquick Gel Extraction Kit; cat no: 28704) and the size of amplicons was checked on agarose gel and were quantified using Qubit Fluorometer. The amplicons were pooled equimolar and purified using AMPure XP beads and quantified using Qubit Fluorometer. After the PCR amplification, the amplified genes were confirmed using Sanger sequencing.

### Nanopore Library Preparation and Sequencing

For Nanopore, the library was prepared using ID sequencing kit (Cat. No SQK-LS 108) as per manufacturer's protocol. The pooled amplicons were subjected to end repair and dA-tailing. The end prepped DNA was quantified using Qubit (Qubit™ dsDNA HS Assay Kit; Cat No: Q32854) followed by ONT adapter ligation. After purification the library was mixed with library loading beads and loaded onto R9 Flowcell to be sequenced for 48 hours on Minion device. Nanopore sequences the entire length of the molecule presented to it regardless of the length of the molecule.

### Super HLA Typing Technology—Complete Sequence Information

In this study, I developed high throughput HLA typing using second and third generation sequencing to obtain full length HLA Class I and Class II genes. The details of HLA genes from both class I (HLA-A, HLA-B and HLA-C) and class II (HLA-DPBI, DQBI and DRBI) was given in Table 1. HLA gene sequences were retrieved from IMGT database (Comparative analyses of Low, Medium and High-Resolution HLA Typing Technologies for Human Populations; Malali Gowda, Sheetal Ambardar, Nutan Dighe, Ashwini Manjunath, Chandana Shankaralingu, Pradeep Hirannaiah, John Harting, Swati Ranade, Latha Jagannathan and Sudhir Krishna, Mar. 9, 2016 J Clin Cell Immunol 7:399) for the designing forward and reverse primer sequences. Using one of the longest sequences as “reference sequence”, other HLA gene sequences from IMGT were aligned to reference, and then most conserved region was selected for designing primer sequences. The amplified PCR products from these primer pairs were amplified using long range Taq polymerase enzyme. The band length was confirmed on agarose gel (FIG. 1B) and sequence identified was confirmed by Sanger sequencing. The sequences obtained from primer pairs for class I and class II HLA genes were matched with the respective HLA genes. Subsequently, PCR amplicons were sequenced using Oxford Nanopore sequencing.

Super HLA Typing method is end-to-end solution, user friendly and cost effective. This can be adopted for HLA registry typing compared to expensive kits used in the current HLA market. I standardized the amplification of full length HLA genes of class I and IL

Third, I have developed Super HLA Typing kit which includes bioinformatics. I integrated bioinformatics tools and developed the allele calling pipeline.

The current HLA-markets in the field of organ transplantation are providing HLA typing services using low resolution typing, incomplete haplotype, which is not sufficient for finding accurate HLA match between donor(s) and recipient.

### EXAMPLES

### Construction of Primers, PCR Amplification, Sequencing and Database Analysis of HLA

### Example 1

### Gene Information

The HLA genes information of HLA-A, HLA-B, HLA-C, HLA-DRBI, HLA-DOBI, HLA-DPBI were obtained from publicly available international databases such as IMGT/HLA (hltp://wVI'N.ebi.ac.uk/imgt/hla/) and NCBI (http://www.ncbi.nlm.nih.gov/gv/mhc/main.fcgi?cmd=init).

### Example 2

### Primer Designing Procedure

Primers for HLA genes were designed to capture full length of HLA sequence. The primers were validated by in-silico PCR amplification for amplicon length and primer position with the reference sequences.

### Example 3

### Artificial Synthesis of Primer Sequences for HLA Genes

Primers of HLA-A, HLA-B, HLA-C, HLA-DQBI, HLA-DRBI and HLA-DPBI genes were synthesized using chemical method (Optimized Automated Solid Phase Synthesis of Oligonucleotides and Derivatives Gabriel Alvarado Urbina, Gerald Grubler3, Angelika Weilerb, Hartmut Echnerb, Stanka Stoevab, Johann Schernthaner, Waleri Gross, Wolfgang Voelterb 53b, 1051-1068 (1998) Z. Naturforsch)

The novel primer sequences of class I (HLA-A, HLA-B, HLA-C) were diagrammatically depicted in FIG. 1-3 for class I HLA genes and their PCR amplification products are shown in FIG. 4. For Class II genes (HLA-DQBI, HLA-DRBI and HLA-DPBI), the novel primer sequences that are diagrammatically depicted in FIG. 5, 7, 9 for class II HLA genes and their PCR amplification products are shown in FIGS. 6, 8 and 10, respectively.

Primer sequences at the beginning and end of each HLA genes were amplified. The size of amplification corresponds to gene length. This confirms primers of the present invention amplify full length of HLA genes.

### EXAMPLES

### Sequencing of PCR Products

PCR amplified HLA products that were gel purified, were then sequenced using long read third generation sequencing, Oxford Nanopore.

### Example 6

### Sequence Data Analysis

The obtained sequence results were BLAST against HLA reference sequences (NCBI/IMGT). The analysis showed HLA amplicon exactly matched primer position in the reference sequences. This confirmed that synthesized PCR primers were able to amplify all the HLA genes.

### Advantages of the Present Invention

There are six classical HLA genes, which are classified into class I (HLA-A, HLA-B, HLA-C) and class II (HLA-DRBI, HLA-DQBI and HLA-DPBI). The present invention provides sequence information for all 6 HLA genes. Therefore the current invention information will be useful for finding match of organ for transplantation.

The previously established HLA typing methods including sequence specific primer (SSP), sequence specific oligonucleotide (SSO) are based on known alleles of HLA gene sequences (e.g., exon 2 and 3 of class I; and exon 2 of class II). These methods only provide low (serological) resolution data of HLA alleles. These methods only provide low resolution and 2-digits information of HLA alleles. The advantage of present invention is that the present method identifies both known and unknown rare alleles of HLA genes.

The previously established HLA typing method by Sanger Sequencing provides limited regions of HLA gene sequences (e.g., exon 2 and 3 of class I, and exon 2 of class II). But this method has certain disadvantages such as low resolution, restriction on length of data to 300-500 bases and is associated with low accuracy, high cost per sample and provides low resolution (2-digit) for HLA genes. Moreover, 41% of HLA-A and 24% of HLA-B alleles is reported to be ambiguous using Sanger Sequencing (Adams et al. 2004). Current invention can provide a high resolution, full-length sequence of HLA genes.

In the international IMGT database (ImMunoGeneTics) only less than 10% and 5% of the HLA sequences contain full length (UTR+EXON+INTRON) sequences for HLA class I and II, respectively. The present invention provides full length gene sequence information.

The advent of second generation sequencing primers developed in prior art (EP2599877A1) claim that they have primers for short read sequencing for HLA-A, B, HLA-C and HLA-DQBI gene. But this invention many not resolve the haplotypes of HLA genes. The current application can provide complete haplotype information for class I (HLA-A, B, C) and class II (DOBI, DRBI and DPBI) genes.

The full length information of HLA genes and 8-digits of HLA alleles are considered to be gold standard approach to identify donors for bone marrow transplantation for the cancer, Thalassemia patients or any organ or any blood disorders.

Current method provides one time solution as health record of a person including autoimmune diseases, drug hypersensitivity and allergy responses and infectious diseases (viral and bacterial).

The Full-length HLA information from current invention will be useful to trace the pedigree of a family. This will be useful for the court to judge parental information during paternity testing, sexually assaulted culprits, etc.

The present invention develops a kit of HLA genes amplification and sequencing using single molecule sequencing.

As per the market survey, there exists a high cost for typing per sample even for low resolution HLA typing. The present super high-resolution invention has substantially reduced cost of HLA typing. This invention is the most cost effective method to date for HLA class I and II genes.

This invention may applied to wide medical and forensic applications.

TABLE 1
HLA Primer Sequences for amplification of full
length HLA genes.

| | |
|---|---|
| | SEQ ID |
| Sequence | No. |

HLA-A MG PCR 01

| | | |
|---|---|---|
| MG_PCR_01_F | CAGAGGGGTCAGGGCGAA | 1 |
| MG_PCR_01_R | ACAGCTCAGTGCACCATGAAG | 2 |

HLA-B MG PCR 02

| | | |
|---|---|---|
| MG_PCR_02_F | CAGACAGTGTGACAAAGAGGCT | 3 |
| MG_PCR_02_R | ATGGGAACAGGGGTCACAGTG | 4 |

HLA-C MG PCR 03

| | | |
|---|---|---|
| MG_PCR_03_F | CAGGCACACAGTGTGACAAAGATG | 5 |
| MG_PCR_03_R | GAGGGAACACAGGTCAGTGTG | 6 |

HLA-DRB1 MG PCR 04
DRB1 amplicon 1 (MG PCR 04 01)

| | | |
|---|---|---|
| MG_PCR_04_01_F | TGAAAGATCMYGGTGCCTTCAT | 7 |
| MG_PCR_04_01_R | GTGTGACMTTYTGCATGAGYAGT | 8 |

DRB1 amplicon 2 (MG PCR 04 02)

| | | |
|---|---|---|
| MG_PCR_04_02_F | GACTATTTAGGADCAYAGCAGGGAATTC | 9 |
| MG_PCR_04_02_R | GTKCWCCACTTGGCACCTAT | 10 |

DRB1 amplicon 3 (MG PCR 04 03)

| | | |
|---|---|---|
| MG_PCR_04_03_F | CAYACACCCTTCRTGYAATCTCTGA | 11 |
| MG_PCR_04_03_R | CAGTAGCAACCAGGTCCKGAG | 12 |

HLA-DQB1 MG PCR 05
DQB1 amplicon 1 (MG PCR 05 01)

| | | |
|---|---|---|
| MG_PCR_05_01_F | GYGTGTCTAAGACAACAGCAGTAA | 13 |
| MG_PCR_05_01_R | ACATRCCAGTCAAWGTGTCAGRTAC | 14 |

DBQ1 amplicon 2 (MG PCR 05 02)

| | | |
|---|---|---|
| MG_PCR_05_02_F | CAGGRCATYCAGCAATTACAGTTG | 15 |
| MG_PCR_05_02_R | TCCCTAGCATCTGGAAAGGTGATG | 16 |

DQB1 amplicon 3 (MG PCR 05 03)

| | | |
|---|---|---|
| MG_PCR_05_03_F | ATTAGGAAYGGTGACTGGACYTTC | 17 |
| BGC_PCR_05_03_F | ACACAGRCAGTYGGGAATTCTG | 18 |

HLA-DPB1 MG PCR 06
DPB1 amplicon 1 (MG PCR 06 01)

| | | |
|---|---|---|
| MG_PCR_06_01_F | CTGTAGATGGGCCAGCAGAAT | 19 |
| MG_PCR_06_01_R | TCAGTCTGATTGGTGCCTCATG | 20 |

DPB1 amplicon 2 (MG PCR 06 02)

| | | |
|---|---|---|
| MG_PCR_06_02_F | AGATGAGCAGAACAATCACAGCAC | 21 |
| MG_PCR_06_02_R | GTCAGCCTCTTCCCTCAATGTG | 22 |

DPB1 amplicon 3 (MG PCR 06 03)

| | | |
|---|---|---|
| MG_PCR_06_03_F | AGTAGTGACAATTCCAGGGTGKATG | 23 |
| MG_PCR_06_03_R | TGARGGTCTGTCCTGGARCCAG | 24 |

Wherein the letters represented as bases are as following:

### R is A or G; Y is C or T; M is A or C; K is G or T; S is C or G; W is A or T; B is C or

### G or T; D is A or G or T; H is A or C or T; V is A or C or G and N is A or C or G or T.

### Details of Class IHLA Gene Sequence Position

TABLE 2
HLA-A gene sequence position

| Exon | | Intron | |
|---|---|---|---|
| physical | | physical | |
| coordinates | Exon length | coordinates | Intron length |
| 5′UTR | 101 | | |
| (1..101) | | | |
| 101..173 | 73 | 174..303 | 130 |
| 304..573 | 270 | 574..814 | 241 |
| 815..1090 | 276 | 1019..1690 | 672 |
| 1691..1966 | 276 | 1967..2065 | 99 |
| 2066..2182 | 117 | 2183..2620 | 438 |
| 2621..2653 | 33 | 2654..2795 | 142 |
| 2796..2843 | 48 | 2844..3012 | 169 |
| 3013..3017 | 5 | 3018..3291 | 273 |
| | | (3′UTR) | |

TABLE 3
HLA-B gene sequence position

| Exon | | Intron | |
|---|---|---|---|
| physical | | physical | |
| coordinates | Exon length | coordinates | Intron length |
| 5′UTR | 301 | | |
| (1..301) | | | |
| 302..373 | 72 | 374..502 | 129 |
| 503..772 | 270 | 773..1022 | 250 |
| 1023..1298 | 276 | 1299..1871 | 573 |
| 1872..2147 | 276 | 2148..2251 | 104 |
| 2252..2368 | 117 | 2369..2809 | 441 |
| 2810..2842 | 33 | 2843..2948 | 106 |
| 2949..2992 | 44 | 2993..3384 | 392 |
| | | (3′UTR) | |

TABLE 4
HLA-C gene sequence position

| Exon | | Intron | |
|---|---|---|---|
| physical | | physical | |
| coordinates | Exon length | coordinates | Intron length |
| (1..344 | 73 | 418..546 | 128 |
| 5′UTR) | | | |
| 345..417 | | | |
| 547..816 | 270 | 817..1062 | 250 |
| 1063..1338 | 276 | 1339..1925 | 574 |
| 1926..2201 | 276 | 2201..2322 | 103 |
| 2323..2442 | 120 | 2443..2881 | 440 |
| 2882..2914 | 33 | 2915..3021 | 106 |
| 3022..3069 | 48 | 3070..3232 | 163 |
| 3233..3238 | 5 | 3239..3457 | |
| | | (3′UTR) | |

### Details of Class II HLA Genes Sequence Position

TABLE 5
HLA-DRBI gene sequence position

| Exon | | Intron | |
|---|---|---|---|
| physical | | physical | |
| coordinates | Exon length | coordinates | Intron length |
| 1..595 | 595 | | |
| (UTR) | | | |
| 596..695 | 100 | 696..11001 | 10305 |
| 11002..11271 | 270 | 11272..13506 | 2234 |
| 13507..13788 | 282 | 13789..14485 | 696 |
| 14486..14596 | 111 | 14597..15071 | 474 |
| 15072..15095 | 24 | 15096..15784 | 688 |
| 15785..15798 | 14 | 15785..16039 | 241 |
| | | (3′UTR) | |

TABLE 6
HLA-DQBI gene sequence position

| Exon | | Intron | |
|---|---|---|---|
| physical | | physical | |
| coordinates | Exon length | coordinates | Intron length |
| (1..529- | 109 | 639..2082 | 1444 |
| 5′UTR) | | | |
| 530..638 | | | |
| 2083..2352 | 270 | 2353..4882 | 2530 |
| 4883..5164 | 282 | 5165..5679 | 515 |
| 5680..5790 | 111 | 5789..6893 | 1105 |
| 6894..6907 | 14 | 6908..7111 | 204 |
| | | (3′UTR) | |

TABLE 7
HLA-DPBI gene sequence position

| Exon | | Intron | |
|---|---|---|---|
| physical | | physical | |
| coordinates | Exon length | coordinates | Intron length |
| (1..366- | 100 | 467..5001 | 4535 |
| 5′UTR) | | | |
| 367..466 | | | |
| 5002..5265 | 264 | 5266..9215 | 3950 |
| 9216..9497 | 282 | 9496..10044 | 549 |
| 10045..10155 | 111 | 10156..10484 | 329 |
| 10485..10504 | 20 | 10505..11461 | 957 |
| | | (3′UTR) | |

1. A method for super HLA typing, gene sequencing and analysis comprising:
(a) designing and synthesizing a set of forward and reverse primers for full length amplification of HLA gene class I (HLA-A, B, C) and class II (HLA DQBI, DRBI, DPBI) selected from group consisting of SEQ ID 1 to 24;
(b) amplifying the test sample HLA gene by using the set of primer synthesized at step (a) to obtain a PCR amplicon;
(c) separating the PCR amplicon obtained at step (b);
(d) sequencing of the separated amplicon of step (c); and
(e) analyzing the sequences generated in step (d) by matching with HLA gene from 5′ UTR to 3′ UTR.

2. The method of claim 1 wherein the primers for super HLA genes are designed by using one of the longest sequences as a reference sequence, other HLA gene sequences from IMGT were aligned to reference, and then the most conserved region was selected for designing primer sequence.

3. The method of claim 1 wherein the amplifications carried with long range Taq polymerase enzyme and the primers were designed in a way that all the primer pairs for class I and class II HLA genes, class I (HLA-A, B, C) and class II (HLA-DRBI, DQBI, DPBI) have common annealing temperature for PCR amplification.

4. The method of claim 1 wherein the set of primers for amplification of HLA-A gene selected are SEQ ID No. 1 and SEQ ID No 2.

5. The method of claim 1 wherein the set of primers for amplification of HLA-B gene selected are SEQ ID No. 3 and SEQ ID No 4.

6. The method of claim 1 wherein the set of primers for amplification of HLA-C gene selected are SEQ ID No. 5 and SEQ ID No 6.

7. The method of claim 1 wherein the set of primers for amplification of HLA-DRBI gene selected are SEQ ID No. 7 to SEQ ID No 12.

8. The method of claim 1 wherein the set of primers for amplification of HLA-DQBI gene selected are SEQ ID No. SEQ ID No. 13 to SEQ ID No 18

9. The method of claim 1 wherein the set of primers for amplification of HLA-DPBI gene selected are SEQ ID No. SEQ ID No. 19 to SEQ ID No 24.

10. The method of claim 1 wherein the obtained sequences were subjected to homology search in biological sequence database to find the best match.

11. Primers for super HLA typing gene sequencing and analysis comprising sequences selected from a group consisting of SEQ ID No. 1 to 24.

12. A kit for super HLA gene sequencing and analysis comprising:
a set of forward and reverse primers for full length amplification of HLA gene class I (HLA-A, B, C) and class II (HLA DQBI, DRBI, DPBI) selected from group consisting of SEQ ID 1 to 24; and
an integrated bioinformatics tools along with the HLA-allele calling.


SEQUENCE LISTING (24 sequences)

SEQ ID NO: 1 - DNA, 18 bp, Artificial Sequence
  Forward primer for Homo sapiens gene HLA-A
cagaggggtcagggcgaa

SEQ ID NO: 2 - DNA, 21 bp, Artificial Sequence
  Reverse primer for Homo sapiens gene HLA-A
acagctcagtgcaccatgaag

SEQ ID NO: 3 - DNA, 22 bp, Artificial Sequence
  Forward primer for Homo sapiens gene HLA-B
cagacagtgtgacaaagaggct

SEQ ID NO: 4 - DNA, 21 bp, Artificial Sequence
  Reverse primer for Homo sapiens gene HLA-B
atgggaacaggggtcacagtg

SEQ ID NO: 5 - DNA, 24 bp, Artificial Sequence
  Forward primer for Homo sapiens gene HLA-C
caggcacacagtgtgacaaagatg

SEQ ID NO: 6 - DNA, 21 bp, Artificial Sequence
  Reverse primer for Homo sapiens gene HLA-C
gagggaacacaggtcagtgtg

SEQ ID NO: 7 - DNA, 22 bp, Artificial Sequence
  Forward primer for Homo sapiens gene HLA-DRB1 Amplicon1
tgaaagatcmyggtgccttcat

SEQ ID NO: 8 - DNA, 23 bp, Artificial Sequence
  Reverse primer for Homo sapiens gene HLA-DRB1 Amplicon1
gtgtgacmttytgcatgagyagt

SEQ ID NO: 9 - DNA, 28 bp, Artificial Sequence
  Forward primer for Homo sapiens gene HLA-DRB1 Amplicon2
gactatttaggadcayagcagggaattc

SEQ ID NO: 10 - DNA, 20 bp, Artificial Sequence
  Reverse primer for Homo sapiens gene HLA-DRB1 Amplicon2
gtkcwccacttggcacctat

SEQ ID NO: 11 - DNA, 25 bp, Artificial Sequence
  Forward primer for Homo sapiens gene HLA-DRB1 Amplicon3
cayacacccttcrtgyaatctctga

SEQ ID NO: 12 - DNA, 21 bp, Artificial Sequence
  Reverse primer for Homo sapiens gene HLA-DRB1 Amplicon3
cagtagcaaccaggtcckgag

SEQ ID NO: 13 - DNA, 24 bp, Artificial Sequence
  Forward primer for Homo sapiens gene HLA-DQB1 Amplicon1
gygtgtctaagacaacagcagtaa

SEQ ID NO: 14 - DNA, 25 bp, Artificial Sequence
  Reverse primer for Homo sapiens gene HLA-DQB1 Amplicon1
acatrccagtcaawgtgtcagrtac

SEQ ID NO: 15 - DNA, 24 bp, Artificial Sequence
  Forward primer for Homo sapiens gene HLA-DQB1 Amplicon2
caggrcatycagcaattacagttg

SEQ ID NO: 16 - DNA, 24 bp, Artificial Sequence
  Reverse primer for Homo sapiens gene HLA-DQB1 Amplicon2
tccctagcatctggaaaggtgatg

SEQ ID NO: 17 - DNA, 24 bp, Artificial Sequence
  Forward primer for Homo sapiens gene HLA-DQB1 Amplicon3
attaggaayggtgactggacyttc

SEQ ID NO: 18 - DNA, 22 bp, Artificial Sequence
  Revesrse primer for Homo sapiens gene HLA-DQB1 Amplicon3
acacagrcagtygggaattctg

SEQ ID NO: 19 - DNA, 21 bp, Artificial Sequence
  Forward primer for Homo sapiens gene HLA-DPB1 Amplicon1
ctgtagatgggccagcagaat

SEQ ID NO: 20 - DNA, 22 bp, Artificial Sequence
  Reverse primer for Homo sapiens gene HLA-DPB1 Amplicon1
tcagtctgattggtgcctcatg

SEQ ID NO: 21 - DNA, 24 bp, Artificial Sequence
  Forward primer for Homo sapiens gene HLA-DPB1 Amplicon2
agatgagcagaacaatcacagcac

SEQ ID NO: 22 - DNA, 22 bp, Artificial Sequence
  Reverse primer for Homo sapiens gene HLA-DPB1 Amplicon2
gtcagcctcttccctcaatgtg

SEQ ID NO: 23 - DNA, 25 bp, Artificial Sequence
  Forward primer for Homo sapiens gene HLA-DPB1 Amplicon3
agtagtgacaattccagggtgkatg

SEQ ID NO: 24 - DNA, 22 bp, Artificial Sequence
  Reverse primer for Homo sapiens gene HLA-DPB1 Amplicon3
tgarggtctgtcctggarccag